(19) United States
(12) Patent Application Publication
(10) Pub. No.: US 2018/0264322 A1
(43) Pub. Date: Sep. 20, 2018

(54) Exercise Support Device, Exercise Support Method, and Storage Medium

(71) Applicant: CASIO COMPUTER CO., LTD., Shibuya-ku (JP)

(72) Inventor: Yuki YOSHIHAMA, Tokyo (JP)

(73) Assignee: CASIO COMPUTER CO., LTD., Shibuya-ku (JP)

(21) Appl. No.: 15/917,092

(22) Filed: Mar. 9, 2018

(30) Foreign Application Priority Data
Mar. 17, 2017 (JP) 2017-053491

(51) Int. Cl. A63B 24/00; G06K 9/00

(52) U.S. Cl. CPC A63B 24/0087; A61B 5/6898; A63B 24/0062; G06K 9/00342

(57) ABSTRACT

An exercise support device includes a processor, a memory, and a sensor. The processor executes a program stored in the memory to perform operations including: acquiring, from the sensor, at least one of biological information about a user related to an exercise and exercise information about the user related to the exercise; and acquiring predetermined information in an outdoor activity involving a movement, on the basis of at least one of the biological information and the exercise information having been acquired, wherein the processor is further configured to acquire at least one of the biological information and the exercise information resulting from an exercise performed the user other than an exercise performed by the user as the movement in the outdoor activity.

### CROSS-REFERENCE TO RELATED APPLICATIONS

This application is based upon and claims the benefit of priority under 35 USC 119 of Japanese Patent Application No. 2017-053491 filed on Mar. 17, 2017 the entire disclosure of which, including the description, claims, drawings, and abstract, is incorporated herein by reference in its entirety.

### BACKGROUND OF THE INVENTION

### Field of the Invention

The present invention relates to an exercise support device, an exercise support method, and a storage medium.

### Related Art

Conventionally, a technology is known that supports outdoor activities, such as climbing and trekking, which involve a movement in a course selected by a user. For example, Japanese Unexamined Patent Application Publication No. 2003-14488 discloses an outdoor system in which a user inputs the exercise capacity and the climbing empirical value of the user and utilizes input parameters so as to estimate an estimated required time for climbing.

### SUMMARY OF THE INVENTION

An exercise support device according to an embodiment of the present invention includes a processor, a memory, and a sensor. The processor executes a program stored in the memory to perform operations including acquiring, from the sensor, at least one of biological information about a user related to an exercise and exercise information about the user related to the exercise, and acquiring predetermined information in an outdoor activity involving a movement, on the basis of at least one of the biological information and the exercise information having been acquired, wherein the processor is further configured to acquire at least one of the biological information and the exercise information resulting from an exercise performed by the user other than an exercise performed by the user as the movement in the outdoor activity.

An exercise support method executed by an exercise support device according to an embodiment of the present invention includes a processor and a sensor. The exercise support method causes the processor to execute a program stored in a memory to perform operations including: acquiring, from the sensor, at least one of biological information about a user related to an exercise and exercise information about the user related to the exercise; and acquiring predetermined information in an outdoor activity involving a movement, on the basis of at least one of the biological information and the exercise information having been acquired, wherein the processor is further configured to acquire at least one of the biological information and the exercise information resulting from an exercise performed by the user other than an exercise performed by the user as the movement in the outdoor activity.

A non-transitory computer-readable storage medium stores a program according to an embodiment of the present invention. The program is executable by a computer that comprises a processor, and the program is executable to cause the computer to perform operations including: acquiring, from the sensor, at least one of biological information about a user related to an exercise and exercise information about the user related to the exercise; and acquiring predetermined information in an outdoor activity involving a movement, on the basis of at least one of the biological information and the exercise information having been acquired, wherein the processor is further configured to acquire at least one of the biological information and the exercise information resulting from an exercise performed by the user other than an exercise performed by the user as the movement in the outdoor activity.

### BRIEF DESCRIPTION OF THE DRAWINGS

FIG. 1 is a diagram showing the configuration of an exercise support system according to an embodiment of the present invention;

FIG. 2 is a block diagram showing the configuration of the hardware of a portable terminal;

FIG. 3 is a block diagram showing the configuration of the hardware of a server;

FIG. 4 is a functional block diagram showing a functional configuration for performing data accumulation processing and navigation processing in the functional configuration of the portable terminal;

FIG. 5A is a diagram schematically showing a conception for setting the exercise level of a user, and is a diagram showing criteria for setting an endurance level at the time of work out;

FIG. 5B is a diagram schematically showing the conception for setting the exercise level of the user, and is a diagram showing criteria for setting an endurance level at the time of walking;

FIG. 5C is a diagram schematically showing the conception for setting the exercise level of the user, and is a diagram showing criteria for setting the level of an exercise frequency;

FIG. 5D is a diagram schematically showing the conception for setting the exercise level of the user, and is a diagram showing the conception for setting the exercise level of the user by integrating the attributes of an exercise capacity;

FIG. 6 is a functional block diagram showing a functional configuration for performing navigation information output processing in the functional configuration of the server;

FIG. 7 is a flowchart illustrating the flow of the data accumulation processing which is performed by the portable terminal of FIG. 2 having the functional configuration of FIG. 4;

FIG. 8 is a flowchart illustrating the flow of the navigation processing which is performed by the portable terminal of FIG. 2 having the functional configuration of FIG. 4; and

FIG. 9 is a flowchart illustrating the flow of the navigation information output processing which is performed by the server of FIG. 3 having the functional configuration of FIG. 6.

### DETAILED DESCRIPTION OF THE INVENTION

Embodiments of the present invention will be explained with reference to the drawings.

An embodiment of the present invention will be described below with reference to drawings.

### [Configuration]

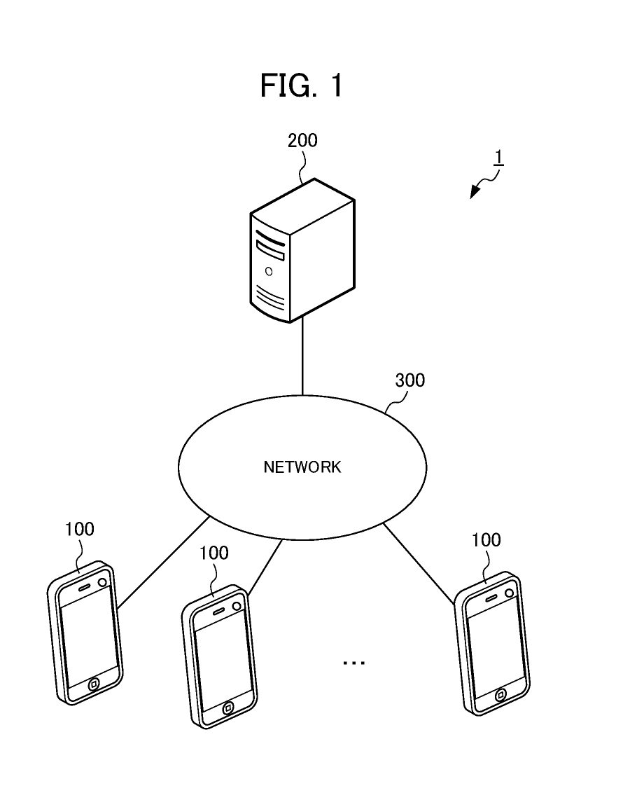

FIG. 1 is a diagram showing the configuration of an exercise support system 1 according to an embodiment of the present invention. In FIG. 1, the exercise support system 1 includes a plurality of portable terminals 100 and a server 200, and the portable terminals 100 and the server 200 are formed so as to be able to communicate with each other through a network 300 such as the Internet. In the present embodiment, the portable terminals 100 are assumed to be respectively used by different users.

### [Hardware Configuration]

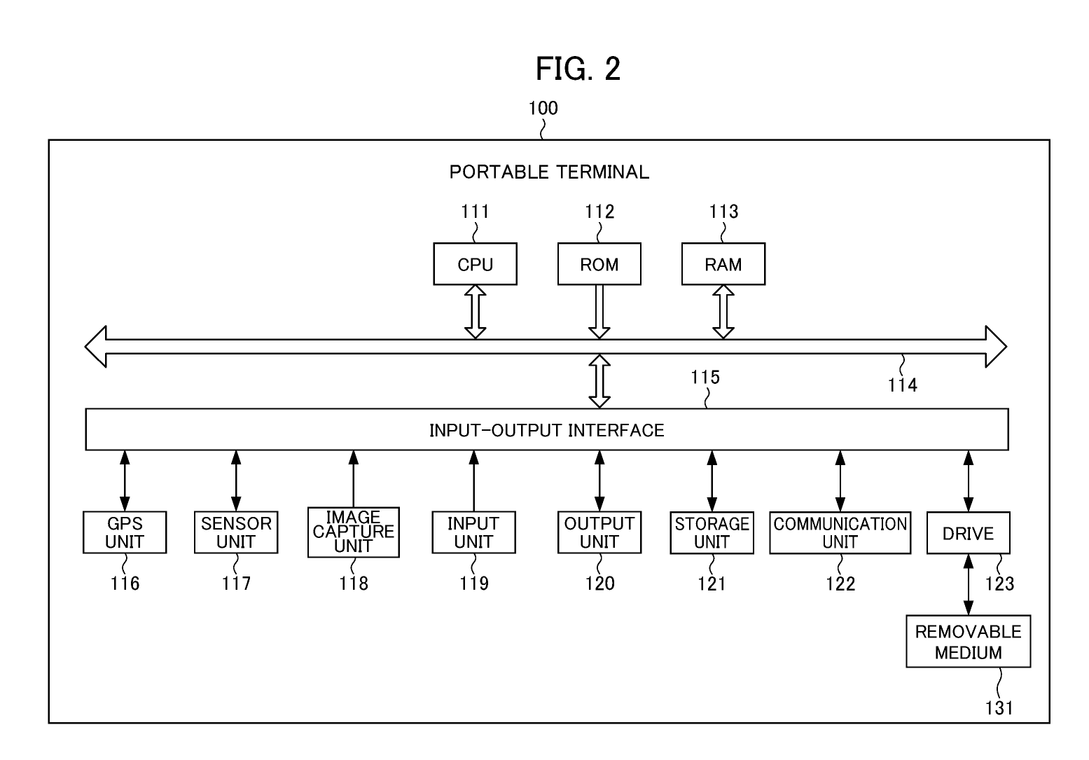

FIG. 2 is a block diagram showing the configuration of the hardware of the portable terminal 100. The portable terminal 100 is an example of an exercise support device according to the present invention, and is formed as, for example, a smartphone.

The portable terminal 100 includes a central processing unit (CPU) 111, a read only memory (ROM) 112, a random access memory (RAM) 113, a bus 114, an input-output interface 115, a global positioning system (GPS) unit 116, a sensor unit 117, an image capture unit 118, an input unit 119, an output unit 120, a storage unit 121, a communication unit 122, and a drive 123.

The CPU 111 executes various types of processing according to a program stored in the ROM 112 or a program loaded from the storage unit 121 into the RAM 113.

Data and the like required by the CPU 111 executing the various processing is stored in the RAM 113 as appropriate.

The CPU 111, the ROM 112, and the RAM 113 are connected to each other via the bus 114. In addition, the input/output interface 115 is also connected to this bus 114. The input-output interface 115 is further connected to the GPS unit 116, the sensor unit 117, the image capture unit 118, the input unit 119, the output unit 120, the storage unit 121, the communication unit 122, and the drive 123.

The GPS unit 16 includes an antenna. The GPS unit 16 receives GPS signals transmitted from a plurality of GPS satellites to acquire position information about the portable terminal 1.

The sensor unit 117 is formed with various types of sensors such as a triaxial acceleration sensor, a triaxial angular velocity sensor, and a triaxial geomagnetic sensor. The sensor unit 117 detects at least the acceleration, the angular velocity, and the geomagnetism that occurs in the portable terminal 100 in accordance with operation of the user, and outputs, as sensor information, the acceleration, the angular velocity, and the geomagnetism thus detected.

The image capture unit 118 includes an optical lens unit and an image sensor, which are not shown. In order to photograph a subject, the optical lens unit is configured by a lens such as a focus lens and a zoom lens for condensing light. The focus lens is a lens for forming an image of a subject on the light receiving surface of the image sensor. The zoom lens is a lens that causes the focal length to freely change in a certain range. The optical lens unit also includes peripheral circuits to adjust setting parameters such as focus, exposure, white balance, and the like, as necessary.

The image sensor is configured by an optoelectronic conversion device, an AFE (Analog Front End), and the like. The optoelectronic conversion device is constituted by an optical sensor such as an optoelectronic conversion device of a CMOS (Complementary Metal Oxide Semiconductor) type. A subject image is incident upon the optoelectronic conversion device through the optical lens unit. The optoelectronic conversion device optoelectronically converts (i.e. captures) the image of the subject, accumulates the resultant image signal for a predetermined period of time, and sequentially supplies the image signal as an analog signal to the AFE. The AFE executes a variety of signal processing such as A/D (Analog/Digital) conversion processing of the analog signal. The variety of signal processing generates a digital signal that is output as an output signal from the image capture unit 118. The output signal of the image capture unit 118 will be hereinafter referred to as “captured image”. The data of the captured image are provided to the CPU 111 and an image processing unit and the like, not shown.

The input unit 119 is constituted by various buttons, microphones, and the like, and inputs a variety of information in accordance with instruction operations by the user. The output unit 120 is constituted by a display, a speaker, and the like, and outputs images and sound. The storage unit 121 is constituted by DRAM (Dynamic Random Access Memory) or the like, and stores data of various images. The communication unit 122 controls communication with a different apparatus (not shown in the drawings) via the network 300 including the Internet.

A removable medium 131 composed of a magnetic disk, an optical disk, a magneto-optical disk, a semiconductor memory or the like is loaded in the drive 123, as necessary. Programs that are read via the drive 123 from the removable medium 131 are installed in the storage unit 121, as necessary. Like the storage unit 121, the removable medium 131 can also store a variety of data stored in the storage unit 121.

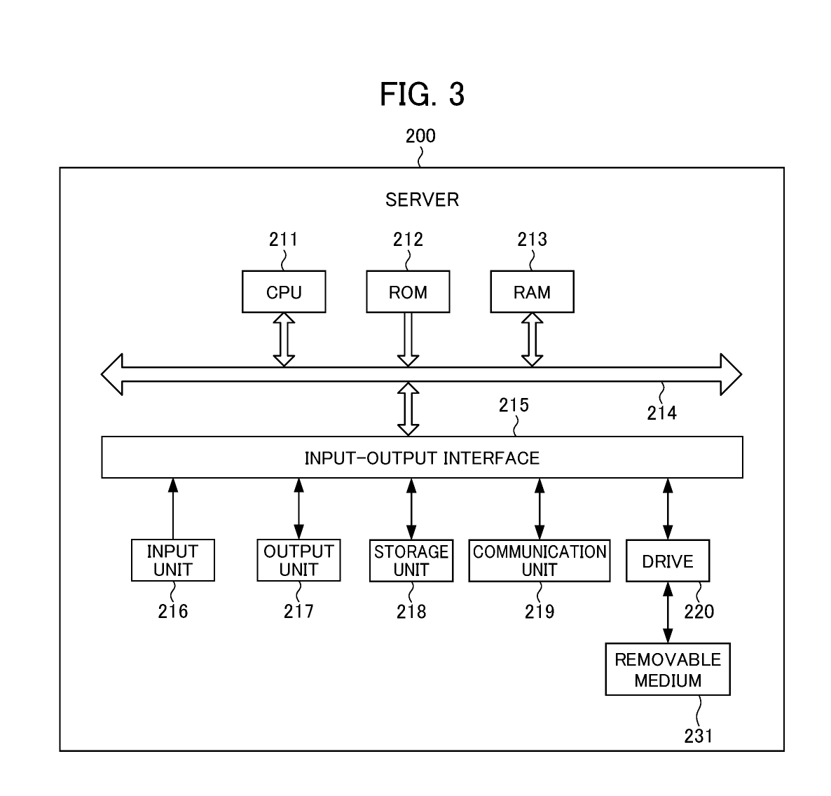

FIG. 3 is a block diagram showing the configuration of the hardware of the portable server 200. The server 200 is formed as, for example, a server computer. The server 200 includes a CPU 211, a ROM 212, a RAM 213, a bus 214, an input-output interface 215, an input unit 216, an output unit 217, a storage unit 218, a communication unit 219, and a drive 220.

The CPU 211 executes various types of processing according to a program stored in the ROM 212 or a program loaded from the storage unit 218 into the RAM 213.

Data and the like required by the CPU 211 executing the various processing is stored in the RAM 213 as appropriate.

The CPU 211, the ROM 212, and the RAM 213 are connected to each other via the bus 214. In addition, the input/output interface 215 is also connected to this bus 214. The input-output interface 215 is further connected to the input unit 216, the output unit 217, the storage unit 218, the communication unit 219, and the drive 220.

The input unit 216 is constituted by various buttons, and the like, and inputs a variety of information in accordance with instruction operations by the user. The output unit 217 is constituted by a display, a speaker, and the like, and outputs images and sound. The storage unit 218 is constituted by DRAM or the like, and stores various kinds of data. The communication unit 219 controls communication with a different apparatus via the network 300 including the Internet.

A removable medium 231 composed of a magnetic disk, an optical disk, a magneto-optical disk, a semiconductor memory or the like is loaded in the drive 220, as necessary. Programs that are read via the drive 220 from the removable medium 231 are installed in the storage unit 218, as necessary. Like the storage unit 218, the removable medium 231 can also store a variety of data such as data of images stored in the storage unit 218.

### [Functional Configuration]

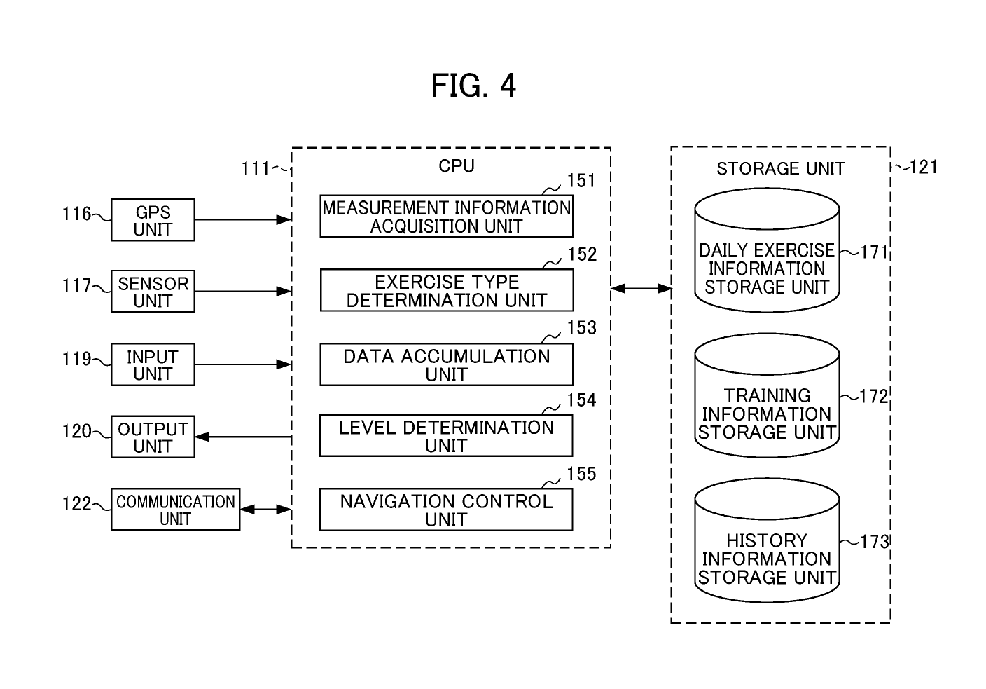

FIG. 4 is a functional block diagram showing a functional configuration for performing data accumulation processing and navigation processing in the functional configuration of the portable terminal 100. The data accumulation processing is a series of processing steps in which user information including the biological information of the user at the time of exercise and information (such as an exercise time and the type of traveling route) on exercise other than the biological information is acquired and accumulated. The navigation processing is a series of processing steps in which based on navigation information output by the server 200 according to the user information, navigation information on a predetermined outdoor activity such as climbing or trekking is displayed.

When the data accumulation processing and the navigation processing are performed, a measurement information acquisition unit 151, an exercise type determination unit 152, a data accumulation unit 153, a level determination unit 154 and a navigation control unit 155 are caused to perform functions in the CPU 111. In one region of a storage unit 121, a daily exercise information storage unit 171, a work out information storage unit 172 and a history information storage unit 173 are set.

In the daily exercise information storage unit 171, the biological information of the user which is detected when the user of the portable terminal 100 performs an exercise (such as walking or the elevation of stairs for movements such as commuting) in daily life is stored so as to be associated with exercise information. Here, the biological information includes a heart rate, a blood pressure and a breathing rate per unit time. The exercise information includes information indicating the type of exercise such as walking or the elevation of stairs and the intensity and the amount of exercise. The intensity of exercise can be indicated by parameters such as a movement speed and an acceleration, and furthermore, in the present embodiment, an exercise intensity (=heart rate/maximum heart rate) serving as an index for indicating endurance is also used as a parameter for indicating the intensity of exercise. The amount of exercise can be indicated by parameters such as the duration of exercise, a movement distance, the number of steps and consumed calories. Hereinafter, information stored in the daily exercise information storage unit 171 is collectively referred to as “daily exercise information” as necessary.

In the work out information storage unit 172, the biological information of the user which is detected when the user of the portable terminal 100 performs work out is stored so as to be associated with the exercise information. Hereinafter, information stored in the work out information storage unit 172 is collectively referred to as “work out information” as necessary.

In the history information storage unit 173, the history information of the user when the user of the portable terminal 100 performs the predetermined outdoor activity such as climbing or trekking is stored so as to be associated with the details of the outdoor activity. For example, in the history information storage unit 173, the history information such as a movement speed and a movement time in a climbing course when the user of the portable terminal 100 actually performs climbing is stored. In the present embodiment, the biological information of the user which is detected when the user of the portable terminal 100 performs the predetermined outdoor activity is also included in the history information. Hereinafter, information stored in the history information storage unit 173 is collectively referred to as “activity history information” as necessary.

The measurement information acquisition unit 151 acquires the position information acquired by the GPS unit 116 and the results of detections of various types of sensors included in the sensor unit 117 (hereinafter, these are collectively referred to as “measurement information” as necessary). The measurement information acquisition unit 151 sequentially acquires the sensor information at a predetermined interval (for example, an interval of one second).

The exercise type determination unit 152 determines, based on the measurement information acquired by the measurement information acquisition unit 151, the type of exercise performed by the user of the portable terminal 100. For example, the exercise type determination unit 152 determines, based on a place and a time at which the exercise is performed and the details of the exercise, which one of a daily exercise, work out and an outdoor activity the exercise performed by the user of the portable terminal 100 is. As an example, when the place where the exercise is performed is a commuting route, the time when the exercise is performed is a commuting time and the details of the exercise is walking, the exercise performed by the user can be determined to be a daily exercise. The user of the portable terminal 100 may manually input the type of exercise, and based on the details of the operation thereof, the exercise type determination unit 152 may determine the type of exercise performed by the user of the portable terminal 100.

The data accumulation unit 153 stores, according to the type of exercise determined by the exercise type determination unit 152, the measurement information acquired by the measurement information acquisition unit 151 in a predetermined region of the storage unit 121. Specifically, the data accumulation unit 153 stores, according to which one of a daily exercise, work out and an outdoor activity the type of exercise determined by the exercise type determination unit 152 is, the measurement information acquired by the measurement information acquisition unit 151, in the daily exercise information storage unit 171 as daily exercise information, in the work out information storage unit 172 as work out information or in the history information storage unit 173 as activity history information. The data accumulation unit 153 may store, in addition to the measurement information acquired by the measurement information acquisition unit 151, information managed by an activity amount application, information acquired from a work out device and the like, as the daily exercise information, the work out information or the activity history information, in a predetermined region of the storage unit 121.

The data accumulation unit 153 uploads, to the server 200, with predetermined upload timing, the daily exercise information stored in the daily exercise information storage unit 171, the work out information stored in the work out information storage unit 172 and the activity history information stored in the history information storage unit 173 (hereinafter, these are collectively referred to as “user exercise information” as necessary). Specifically, the data accumulation unit 153 uploads the user information within the storage unit 121 to the server 200 with the upload timing such as timing at which a movement in the course of the outdoor activity is completed, timing at which work out is completed or timing during sleeping in daily life.

The level determination unit 154 determines the exercise level of the user based on the daily exercise information stored in the daily exercise information storage unit 171, the work out information stored in the work out information storage unit 172 and the activity history information stored in the history information storage unit 173 (the user exercise information). The exercise level is an index for indicating the current exercise capacity of the user, and for example, based on the user exercise information, the level is individually set for the attributes of an exercise capacity, the levels for the individual attributes are integrated and thus it is possible to set the exercise level of the user. The level determination unit 154 updates the exercise level of the user each time the user exercise information is updated. The level determination unit 154 stores the levels for the attributes of the exercise capacity and the exercise level of the user in the storage unit 121.

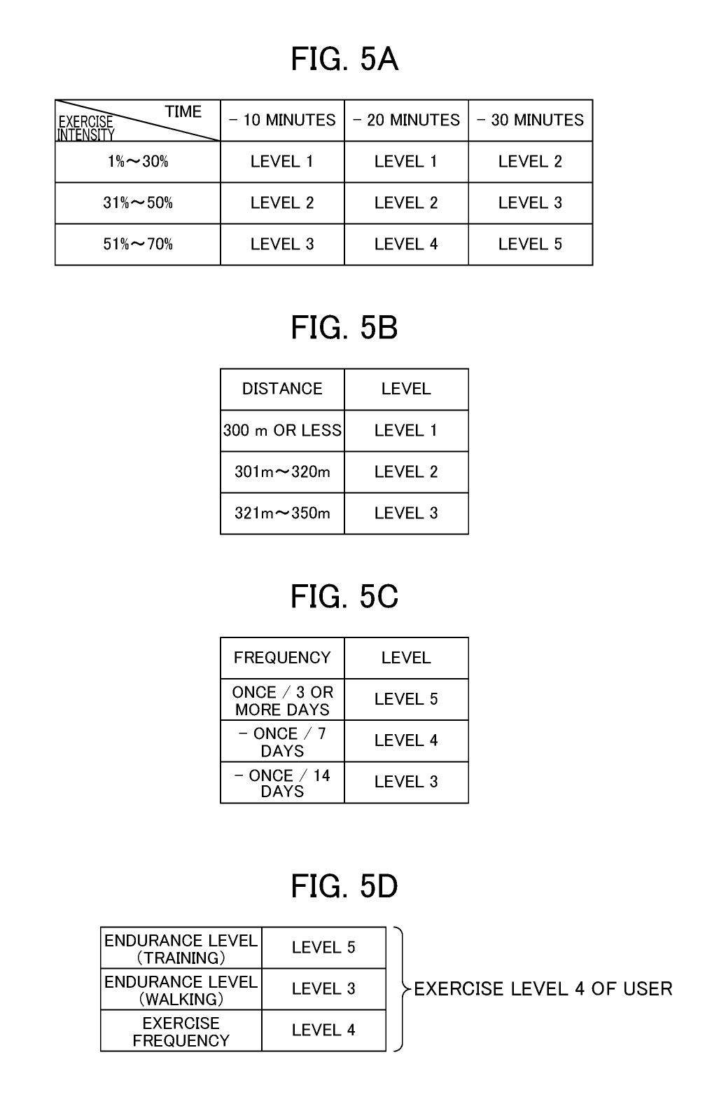

FIGS. 5A to 5D are diagrams schematically showing a conception for setting the exercise level of the user, FIG. 5A is a diagram showing criteria for setting an endurance level at the time of work out, FIG. 5B is a diagram showing criteria for setting an endurance level at the time of walking, FIG. 5C is a diagram showing criteria for setting the level of an exercise frequency and FIG. 5D is a diagram showing a conception for setting the exercise level of the user by integrating the attributes of the exercise capacity.

As shown in FIG. 5A, for the endurance level at the time of work out, the exercise intensity (=heart rate/maximum heart rate) and the possible duration of exercise are used as indices, and endurance levels corresponding to the ranges of the individual values are set. As shown in FIG. 5B, for the endurance level at the time of walking, endurance levels are set according to a possible walking distance per unit time (here, 3 minutes).

Furthermore, as shown in FIG. 5C, for the level of the exercise intensity, the levels of the exercise intensity are set according to the frequency at which exercise at reference exercise intensity (for example, an “exercise intensity of 30%” which corresponds to a light exercise) or more is performed. Then, as shown in FIG. 5D, the levels set for the individual attributes of exercises included in the user exercise information are integrated, and thus the exercise level of the user is set. As a method of integrating the levels set for the individual attributes of exercises included in the user exercise information, for example, the average value of the levels can be used. When the exercise level of the user is set, the average value can be acquired by assigning weights to the individual attributes of exercises or a value (the minimum value or the maximum value) other than the average value can be used.

The navigation control unit 155 performs, based on the navigation information serving as predetermined information acquired from the server 200, processing for controlling navigation when the user performs the outdoor activity. For example, the navigation control unit 155 performs processing in which the navigation information where an estimated required time is set according to the exercise level of the user is acquired from the server 200, in which the navigation information is displayed on a map and in which the result of a comparison between the navigation information and the movement history of the user is sequentially displayed (notified) as the user actually moves. When in the present embodiment, navigation is performed, the user can select which one of the type of navigation based on the user exercise information of the individual user (hereinafter referred to as “individual-based navigation”) and the type of navigation based on the user exercise information of a plurality of users (hereinafter referred to as “multiple user-based navigation”) is used. When the individual-based navigation is selected, the navigation information is generated by use of only the user exercise information of the user who requires the output of the navigation information. On the other hand, when the multiple user-based navigation is selected, the navigation information is generated by use of the user exercise information of the user who requires the output of the navigation information and the user exercise information of the other users.

When an instruction is input by the user, the navigation control unit 155 converts the navigation information acquired from the server 200 into the form of a course planning document (for example, a climbing planning document in climbing), and prints the course planning document from a printer (unillustrated) connected through the network 300 or the like. The course planning document can be printed on a paper medium so as to be submitted to a predetermined submission place or can be submitted, as data in a predetermined form (such as PDF), to the predetermined submission place through the network 300.

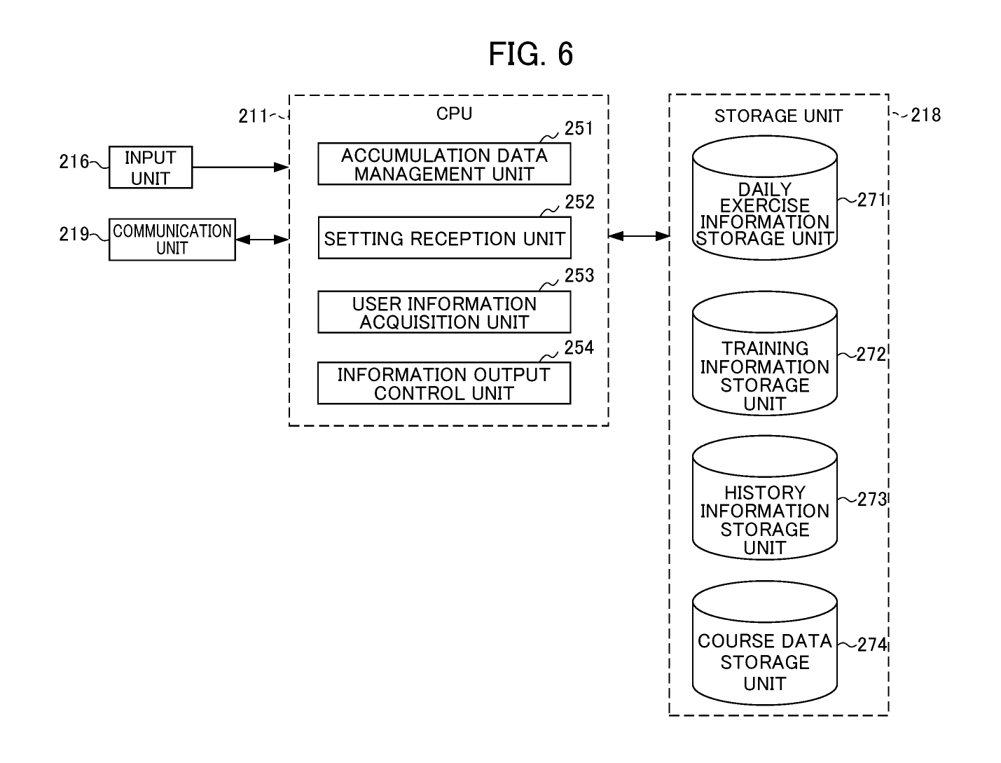

The functional configuration of the server 200 will then be described. FIG. 6 is a functional block diagram showing a functional configuration for performing navigation information output processing in the functional configuration of the server 200. The navigation information output processing is a series of processing steps in which in response to the requirement of an output of navigation information on a specific outdoor activity from the user of the portable terminal 100, an estimated required time for the outdoor activity is set according to the exercise level of the user, and in which information on the course of the outdoor activity and navigation information including the estimated required time are provided.

When the navigation information output processing is performed, as shown in FIG. 6, in the CPU 211, an accumulation data management unit 251, a setting reception unit 252, a user information acquisition unit 253 and an information output control unit 254 function. In one region of the storage unit 218, a daily exercise information storage unit 271, a work out information storage unit 272, a history information storage unit 273 and a course data storage unit 274 are set.

In the daily exercise information storage unit 271, the biological information of users which are detected when the users of a plurality of portable terminals 100 perform exercises in daily life is stored so as to be associated with exercise information. In the work out information storage unit 272, the biological information of the users which are detected when the users of the portable terminals 100 perform work out is stored so as to be associated with the exercise information.

In the history information storage unit 273, the history information of the users when the users of the portable terminals 100 perform the predetermined outdoor activities such as climbing and trekking is stored so as to be associated with the details of the outdoor activities. In the course data storage unit 274, for the courses of the outdoor activities, route information and the standard required times of the courses are stored so as to be associated with each other. For example, the standard required times of the courses of the outdoor activities can be previously set or can be set to the average value to which weights are assigned with consideration given to the exercise levels of the individual users based on the actual results of the users of the portable terminals 100.

The accumulation data management unit 251 respectively stores, for the individual types (the daily exercise information, the work out information and the activity history information), the user exercise information of the users uploaded by a plurality of portable terminals 20 in the daily exercise information storage unit 271, the work out information storage unit 272 and the history information storage unit 273.

The setting reception unit 252 receives the settings of the target course for the outdoor activity and the type of navigation (the individual-based navigation or the multiple user-based navigation). When the multiple user-based navigation is performed, for the target outdoor activities which are not actually performed by the user, the estimated required times of a higher probability can be presented with reference to the user exercise information of the other users. On the other hand, when the individual-based navigation is performed, the estimated required time specific to the characteristic of the user can be presented.

The user information acquisition unit 253 acquires information on the exercise capacity of the user of the portable terminal 100 who requires the output of the navigation information. In the present embodiment, the exercise level of the user stored in the storage unit 121 of the portable terminal 100 is transmitted from the portable terminal 100 to the server 200.

The information output control unit 254 compares, with reference to the information (exercise level) on the exercise capacity of the user who requires the output of the navigation information, in the activity history information of the user, the standard required time for the exercise level and the actual required time of the user, and thereby calculates a user coefficient specific to the user. The user coefficient is a parameter for adjusting a difference between the exercise capacity of the user and the standard exercise capacity in the exercise level of the user. For example, when it takes 6 hours for the user to move in a course in which it takes 5 hours in the case of the standard exercise capacity in the exercise level of the user, the user coefficient of the user is calculated as user coefficient=actual required time/standard required time=1.2. When the history is present in which the user moved in the course of the outdoor activity required for the output of the navigation information, the user coefficient can be calculated from the history information thereof. When the history is not present in which the user moved in the course of the outdoor activity required for the output of the navigation information, the user coefficient can be calculated with reference to the history information of other courses (for example, the other courses in which the distance, the gradient, the type of road surface and the like are common to each other) having similar characteristics to the course.

The information output control unit 254 acquires, from the course data storage unit 274, the data of the course of the outdoor activity required for the output of the navigation information. In the present embodiment, when the individual-based navigation is set, the information output control unit 254 multiplies, in the course of the outdoor activity required for the output of the navigation information, the standard required time corresponding to the exercise level of the user by the user coefficient, and thereby calculates the estimated required time for the user. When the multiple user-based navigation is set, the information output control unit 254 calculates the estimated required time for the user based on a value obtained by multiplying the standard required time corresponding to the exercise level of the user by the user coefficient and the actual required time of other users having the same exercise level as the user in the course of the outdoor activity required for the output of the navigation information. For example, the information output control unit 254 can calculate the estimated required time for the user in the multiple user-based navigation by performing weighted averaging on the value obtained by multiplying the standard required time corresponding to the exercise level of the user by the user coefficient and the average value of the actual required times of the other users having the same exercise level as the user. Then, the information output control unit 254 transmits, to the portable terminal 100, the navigation information in which the estimated required time for the user is set.

### [Operation]

The operation of the exercise support system 1 will then be described.

### [Data Accumulation Processing]

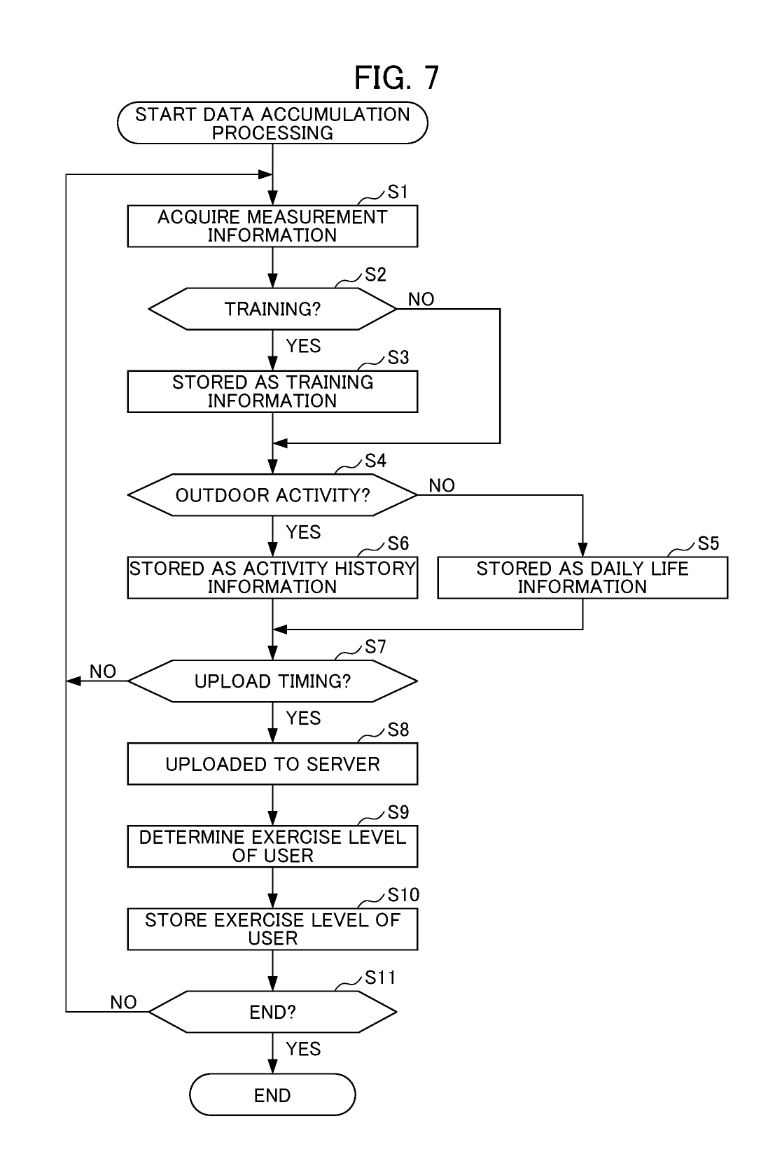

FIG. 7 is a flowchart illustrating the flow of the data accumulation processing which is performed by the portable terminal 100 of FIG. 2 having the functional configuration of FIG. 4. The data accumulation processing is started by performing an operation of providing, through the input unit 119, an instruction to start the data accumulation processing.

In step S1, the measurement information acquisition unit 151 acquires the position information acquired by the GPS unit 116 and the results of detections (the measurement information) of various types of sensors included in the sensor unit 117. In step S2, the exercise type determination unit 152 determines, based on the measurement information acquired by the measurement information acquisition unit 151, whether or not the type of exercise performed by the user of the portable terminal 100 is work out.

When the type of exercise performed by the user of the portable terminal 100 is work out, in step S2, a determination of yes is made, and the processing is transferred to step S3. On the other hand, when the type of exercise performed by the user of the portable terminal 100 is not work out, in step S2, a determination of no is made, and the processing is transferred to step S4. In step S3, the data accumulation unit 153 stores, in the work out information storage unit 172, as the work out information, the measurement information acquired in step S1. In step S4, the exercise type determination unit 152 determines, based on the measurement information acquired by the measurement information acquisition unit 151, whether or not the type of exercise performed by the user of the portable terminal 100 is the predetermined outdoor activity (such as climbing or trekking).

When the type of exercise performed by the user of the portable terminal 100 is not the predetermined outdoor activity, in step S4, a determination of no is made, and the processing is transferred to step S5. On the other hand, when the type of exercise performed by the user of the portable terminal 100 is the predetermined outdoor activity, in step S4, a determination of yes is made, and the processing is transferred to step S6.

In step S5, the data accumulation unit 153 stores, in the daily exercise information storage unit 171, as the daily exercise information, the measurement information acquired in step S1. On the other hand, in step S6, the data accumulation unit 153 stores, in the history information storage unit 173, as the activity history information, the measurement information acquired in step S1.

In step S7, the data accumulation unit 153 determines whether or not it is timing at which the user exercise information is uploaded. When it is timing at which the user exercise information is uploaded, in step S7, a determination of yes is made, and processing is transferred to step S8. On the other hand, when it is not timing at which the user exercise information is uploaded, in step S7, a determination of no is made, and processing is transferred to step S1.

In step S8, the data accumulation unit 153 uploads the user exercise information to the server 200. In step S9, the level determination unit 154 determines the exercise level of the user based on the user exercise information. Here, the levels for the individual attributes in the exercise capacity of the user and the exercise level of the user are calculated. In step S10, the level determination unit 154 stores, in the storage unit 121, the levels for the individual attributes in the exercise capacity and the exercise level of the user. In step S11, the level determination unit 154 determines whether or not an instruction to complete the data accumulation processing is provided.

When an instruction to complete the data accumulation processing is not provided, in step S11, a determination of no is made, and the processing is transferred to step S1. On the other hand, when an instruction to complete the data accumulation processing is provided, in step S11, a determination of yes is made, and the data accumulation processing is completed.

### [Navigation Processing]

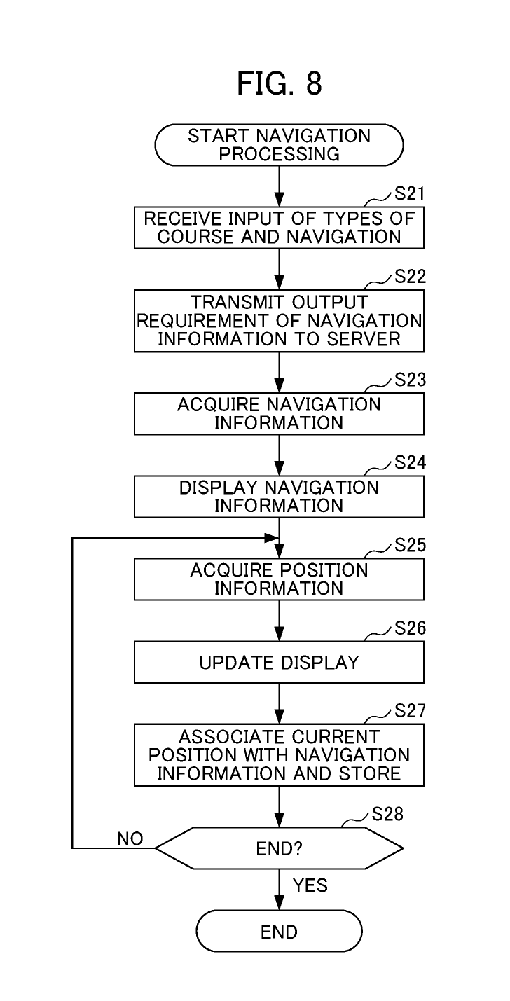

FIG. 8 is a flowchart illustrating the flow of the navigation processing which is performed by the portable terminal 100 of FIG. 2 having the functional configuration of FIG. 4. The navigation processing is started by performing an operation of providing, through the input unit 119, an instruction to start the navigation processing. In step S21, the navigation control unit 155 accepts the input of the target course where the outdoor activity is performed by the user and the type of navigation (the individual-based navigation or the multiple user-based navigation).

In step S22, the navigation control unit 155 transmits, to the server 200, the target course where the accepted outdoor activity is performed and the type of navigation as a requirement for the output of the navigation information. In step S23, the navigation control unit 155 acquires the navigation information from the server 200. In step S24, the navigation control unit 155 displays the received navigation information on the display of the output unit 120.

In step S25, the measurement information acquisition unit 151 acquires the position information of the portable terminal 100. In step S26, the navigation control unit 155 updates the display of the navigation information so as to superimpose and display the current position based on the acquired position information. In step S27, the navigation control unit 155 associates the current position with the navigation information, and stores it in the history information storage unit 173 of the storage unit 121.

In step S28, the navigation control unit 155 determines whether or not an instruction to complete the navigation processing is provided. When an instruction to complete the navigation processing is not provided, in step S28, a determination of no is made, and the processing is transferred to step S25. On the other hand, when an instruction to complete the navigation processing is provided, in step S28, a determination of yes is made, and the navigation processing is completed. A configuration may be adopted in which when the current position of the user coincides with the destination of the course in the navigation information, the navigation control unit 155 automatically completes the navigation processing.

### [Navigation Information Output Processing]

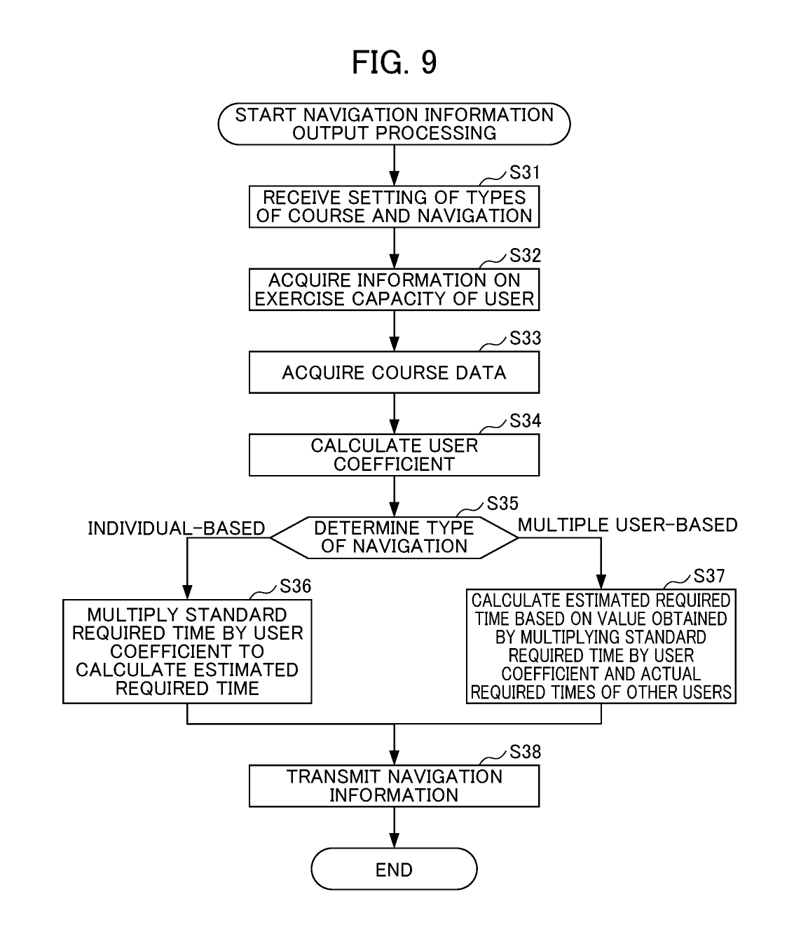

FIG. 9 is a flowchart illustrating the flow of the navigation information output processing which is performed by the server 200 of FIG. 3 having the functional configuration of FIG. 6. The navigation information output processing is started by performing an operation of providing, through the input unit 216, an instruction to start the navigation information output processing. In step S31, the setting reception unit 252 receives the settings of the target course for the outdoor activity and the type of navigation (the individual-based navigation or the multiple user-based navigation).

In step S32, the user information acquisition unit 253 acquires information (exercise level) on the exercise capacity of the user of the portable terminal 100 who requires the output of the navigation information. In step S33, the information output control unit 254 acquires, from the course data storage unit 274, the data of the course of the outdoor activity required for the output of the navigation information. In step S34, the information output control unit 254 compares, with reference to the information (exercise level) on the exercise capacity of the user who requires the output of the navigation information, in the activity history information of the user, the standard required time for the exercise level and the actual required time of the user, and thereby calculates a user coefficient specific to the user.

In step S35, the information output control unit 254 determines which one of the individual-based navigation or the multiple user-based navigation is set (the type of navigation). When the individual-based navigation is set, the processing is transferred to step S36 by the determination in step S35. On the other hand, when the multiple user-based navigation is set, the processing is transferred to step S37 by the determination in step S35.

In step S36, the information output control unit 254 multiplies, in the course of the outdoor activity required for the output of the navigation information, the standard required time corresponding to the exercise level of the user by the user coefficient, and thereby calculates the estimated required time for the user. In step S37, the information output control unit 254 calculates the estimated required time for the user based on the value obtained by multiplying the standard required time corresponding to the exercise level of the user by the user coefficient and the actual required time of other users having the same exercise level as the user in the course of the outdoor activity required for the output of the navigation information. In step S38, the information output control unit 254 transmits, to the portable terminal 100, the navigation information in which the estimated required time for the user is set. After step S38, the navigation information output processing is completed.

By the processing described above, in the exercise support system 1 according to the present embodiment, the portable terminal 100 accumulates the information (the daily exercise information or the work out information) on exercises other than the outdoor activity performed by the user. Then, when the navigation information in the outdoor activity (such as climbing) involving a movement is provided to the portable terminal 100, the server 200 calculates, based on the exercise level of the user set from the daily exercise information or the work out information of the user, the estimated required time (the estimated required time of the course) on the movement of the outdoor activity. Hence, the conditions of the exercise and the work out performed by the user in daily life are reflected, and it is possible to easily acquire and provide the accurate navigation information on the outdoor activity involving a movement.

In the exercise support system 1 according to the present embodiment, when the history (activity history information) in which the outdoor activity was performed by the user in the past is stored, the server 200 generates the navigation information with reference to the activity history information (the activity history information of the same course or similar courses) which can be utilized when the estimated required time is calculated. Hence, the history of the outdoor activity is additionally used, and thus it is possible to provide more accurate navigation information.

In the exercise support system 1 according to the present embodiment, which one of the type of navigation (individual-based navigation) based on the user exercise information of the individual user and the type of navigation (multiple user-based navigation) based on the user exercise information of a plurality of users is used is selected by the user. Then, when the individual-based navigation is selected, the server 200 generates the navigation information by use of only the user exercise information of the user who requires the output of the navigation information. In this case, the estimated required time specific to the characteristic of the user can be presented. On the other hand, when the multiple user-based navigation is selected, the navigation information is generated by use of the user exercise information of the user who requires the output of the navigation information and the user exercise information of the other users by the server 200. In this case, for the target outdoor activities which are not actually performed by the user, the estimated required times of a higher probability can be presented with reference to the user exercise information of the other users.

### [Variation 1]

In the description of the embodiment discussed above, the navigation information output by the information output control unit 254 of the server 200 corresponds to the exercise level of the user and presents the estimated required time of the course. By contrast, the server 200 (the information output control unit 254) has the route setting function of setting a route based on map information, and thereby can present a recommended course to the user either instead of the estimated required time of the course or together with the estimated required time of the course. In this case, the navigation control unit 155 of the portable terminal 100 requests the server 200 to set the route, and thus the information output control unit 254 of the server 200 sets the route. In this way, the navigation information output by the server 200 corresponds to the exercise level of the user (or the user exercise information serving the base thereof), and thus a more appropriate course is presented when the user performs the outdoor activity.

As an example, when the target of the outdoor activity in which the user requires the output of the navigation information is a climbing route, the server 200 compares the exercise level of the user and the level (the degree of difficulty) set for the climbing route (the course of the outdoor activity), and when the exercise level of the user is separated from the level of the climbing route by a set threshold value or more, the server 200 can propose another climbing route (such as a climbing route simpler than that of the same mountain) to the user. The level of the climbing route (the course of the outdoor activity) can be previously set. The level of the climbing route may be automatically set from the exercise levels of a plurality of users who select the climbing route, the actual required time or the like. For example, the level of the climbing route can be set from the average value of the exercise levels of the users who select the climbing route. From a relationship between the actual required time and the exercise level of the user who selects the climbing route, the estimated minimum exercise level required for the selection of the climbing route can be set.

### [Variation 2]

In the embodiment described above, when the navigation information on the outdoor activity is generated, the type of outdoor activity can be selected, and for each type of outdoor activity, the navigation information can be generated with a different algorithm. For example, the setting reception unit 252 of the server 200 accepts the selection of one type from a plurality of types of outdoor activities, and thus the information output control unit 254 can generate the navigation information with reference to the user exercise information and with an algorithm corresponding to the received outdoor activity. As the algorithm corresponding to the outdoor activity, for example, a climbing route algorithm, a trekking algorithm, a cycling algorithm and the like can be previously prepared. Then, even when the same user moves in the same course, a different estimated required time can be calculated depending on which one of a climbing route, a trekking route and a cycling route the user moves in.

### [Variation 3]

In the embodiment and variations 1 and 2 described above, the portable terminal 100 and the server 200 cooperate with each other so as to realize the function of the exercise support system 1. By contrast, the function of the server 200 in the embodiment and variations 1 and 2 described above may be included in the portable terminal 100. In this case, in the navigation control unit 155 of the portable terminal 100, it is possible to realize the route setting function of setting a route based on the map information, the function of generating the navigation information for each type of outdoor activity with a different algorithm, the function of outputting the navigation information for the course of the outdoor activity required by the user and the like. In the portable terminal 100 configured as described above, the generation of various types of navigation information in the embodiment and variations 1 and 2 described above can be performed only by the portable terminal 100.

The portable terminal 100 configured as described above includes the measurement information acquisition unit 151 and the navigation control unit 155. The measurement information acquisition unit 151 acquires, from the sensor, at least one of the biological information and the exercise information of the user related to the exercise. The navigation control unit 155 acquires, based on at least one of the biological information and the exercise information acquired by the measurement information acquisition unit 151, the navigation information in the outdoor activity involving a movement. The measurement information acquisition unit 151 acquires at least one of the biological information and the exercise information resulting from the exercise of the user other than the exercise performed as the movement in the outdoor activity. In this way, the information other than the exercise performed as the movement in the outdoor activity is reflected, and it is possible to easily acquire accurate navigation information on the outdoor activity involving a movement.

The portable terminal 100 also includes the navigation control unit 155. The navigation control unit 155 sets a route with the map information. The navigation control unit 155 acquires the navigation information on the set route. In this way, it is possible to present a more appropriate course (route) when the user performs the outdoor activity.

The navigation control unit 155 sets the route based on at least one of the biological information and the exercise information acquired by the measurement information acquisition unit 151. In this way, it is possible to set a more appropriate course (route) depending on the conditions of the exercise of the user.

The navigation control unit 155 calculates, based on the biological information or the exercise information acquired by the measurement information acquisition unit 151, the required time on the set route. The navigation control unit 155 acquires the calculated required time. In this way, it is possible to acquire a more accurate required time based on the conditions of the exercise of the user.

The navigation control unit 155 selects one outdoor activity from a plurality of outdoor activities. The navigation control unit 155 acquires the navigation information based on the selected outdoor activity. In this way, even when the same user moves in the same course, it is possible to acquire a different required time depending on the selected outdoor activity.

The measurement information acquisition unit 151 acquires biological information in at least one of the daily exercise information and work out information of the user. In this way, it is possible to more accurately grasp the state of the body of the user in the exercise or the work out daily performed by the user and thereby acquire more appropriate navigation information.

The measurement information acquisition unit 151 identifies the exercise level of the user from at least one of the exercise information and the work out information. The navigation control unit 155 acquires the navigation information based on the exercise level. In this way, it is possible to classify the exercise capacity of the user and thereby acquire more appropriate navigation information according to the classification.

The navigation control unit 155 acquires the biological information or the exercise information of the user with a sensor fitted to the user or acquires the biological information or the exercise information of the user by analysis of an image obtained by sensing the user. In this way, based on various signs appearing in the body of the user, it is possible to accurately grasp the conditions of the user at the time of exercise.

The portable terminal 100 includes the level determination unit 154. The level determination unit 154 determines the empirical value of the user based on the history of the outdoor activity performed by the user in the past. The navigation control unit 155 further acquires the navigation information based on the empirical value determined by the level determination unit 154. In this way, the history of the outdoor activity is additionally used, and thus it is possible to acquire more accurate navigation information.

The navigation control unit 155 acquires the navigation information in the outdoor activity of the user involving a movement based on at least one of biological information and exercise information acquired for other users. In this way, for the target outdoor activities which are not actually performed by the user, the navigation information of a higher probability can be acquired with reference to, for example, the biological information or the exercise information of the other users.

The present invention is not limited to the embodiment described above, and as long as the object of the present invention can be achieved, variations, modifications and the like are included in the present invention.

For example, in the embodiment described above, calories taken in daily life may be included in the daily exercise information, and the daily exercise information may be acquired. In this case, whether or not the calories taken are appropriate can be used as an element for determining the exercise level.

When in the embodiment described above, the navigation information is generated, related information such as weather forecast may be reflected, and the estimated required time may be set. In this case, for example, the estimated required time can be calculated by multiplying the standard required time by the user coefficient and further multiplying it by a compensation coefficient (for example, in case of rain, it is set to 1.2 times) of the related information such as weather forecast.

When in the embodiment described above, the level determination unit 154 determines the exercise level of the user, the level determination unit 154 may perform feedback processing by feeding back the degree of agreement between navigation information on a course where the estimated required time is set based on the exercise level set in the past and the actual activity history information of the user when the user moves in the course such that criteria are changed.

When in the embodiment described above, the information output control unit 254 calculates the user coefficient, the information output control unit 254 may perform feedback processing by comparing, in a plurality of users, the degrees of agreement between navigation information on a course where the estimated required time is set based on the exercise level set in the past and the actual activity history information of the individual users when the users move in the course such that the calculation criteria of the user coefficient are changed. The user exercise information and the activity history information (the actual history for the estimated required time) may be analyzed, and the result of the analysis may be reflected on an algorithm for calculating the estimated required time. In this way, as the exercise support system 1 is utilized more, a more accurate estimated required time can be presented.

In the embodiment described above, the width of a variation in the estimated required time (the expectation of a variation in the required time) may be calculated according to the exercise level of the user. For example, the width of the variation can be calculated such that as the exercise level is higher, the actual time is closer to the estimated required time whereas as the exercise level is lower, a difference between the actual time and the estimated required time is increased.

In the embodiment described above, the measurement information acquisition unit 151 may not only acquire the measurement information from the GPS unit 116 or the sensor unit 117 included in the portable terminal 100 but also acquire, through the communication unit 122 or the like, the measurement information acquired in another device.

When in the embodiment described above, the user exercise information is acquired, not only the user exercise information can be detected with a sensor included in the portable terminal 100 or another wearable terminal but also an image such as the facial expression or the behavior of the user is sensed with the portable terminal 100 or an image sensing device, and thus the user exercise information can be acquired from the result of the analysis of the image. For example, when as a result of the analysis of an image of the face of the user, the facial expression is determined to be a “painful expression”, the user exercise information can be regarded as indicating that a high-intensity exercise is performed (the intensity of an exercise is high-level).

Although in the embodiment described above, the activity history information of the user who requires the output of the navigation information is used for the calculation of the user coefficient, there is no limitation to this configuration. For example, the activity history information of the user is regarded as indicating the empirical value (level) of the outdoor activity of the user, and thus the level determination unit 154 may calculate the exercise level of the user based on the activity history information.

For example, the functions included in the portable terminal 100 and the server 200 in the embodiment described above can be mounted on one device or can be divided so as to be mounted on three or more devices, that is, various types of system forms can be adopted as long as the functions of the exercise support system 1 are realized as a whole. For example, although in the embodiment described above, the CPU 111 within the portable terminal 100 performs the processing shown in the flowcharts of FIGS. 7 and 8, a configuration can be adopted in which data is transmitted through the communication unit 122 to the server 200, in which the server 200 performs computation processing, in which the result thereof is transmitted to the portable terminal 100 and in which the navigation information is displayed on the display of the output unit 120. Although in the embodiment described above, the CPU 211 within the server 200 performs the processing shown in the flowchart of FIG. 9, the processing can also be performed in the portable terminal 100.

Although in the embodiment described above, a smartphone is adopted as an example for explaining the exercise support device to which the present invention is applied, but the embodiment is not limited thereto. For example, the present invention can be applied to electronic devices in general that include a notification function for obtaining user exercise information and notifying information. For example, the present invention can be applied to a notebook type personal computer, a tablet type terminal, a camcorder, a portable type navigation device, a cellular phone, a wrist type terminal, a portable game device, and the like.

The processing sequence described above can be executed by hardware, and can also be executed by software. In other words, the hardware configuration of FIG. 4 is merely an illustrative example, and the present invention is not particularly limited thereto. More specifically, the types of functional blocks employed to realize the above-described functions are not particularly limited to the examples shown in FIG. 4, so long as the portable terminal 100 can be provided with the functions enabling the aforementioned processing sequence to be executed in its entirety. A single functional block may be constituted by a single piece of hardware, a single installation of software, or a combination thereof. The functional configurations of the present embodiment are realized by a processor executing arithmetic processing, and processors that can be used for the present embodiment include a unit configured by a single unit of a variety of single processing devices such as a single processor, multi-processor, multi-core processor, etc., and a unit in which the variety of processing devices are combined with a processing circuit such as ASIC (Application Specific Integrated Circuit) or FPGA (Field-Programmable Gate Array).

In the case of having the series of processing executed by software, the program constituting this software is installed from a network or recording medium to a computer or the like. The computer may be a computer equipped with dedicated hardware. In addition, the computer may be a computer capable of executing various functions, e.g., a general purpose personal computer, by installing various programs.

The storage medium containing such a program can not only be constituted by the removable medium 131 of FIG. 4 distributed separately from the device main body for supplying the program to a user, but also can be constituted by a storage medium or the like supplied to the user in a state incorporated in the device main body in advance. The removable medium 131 is composed of, for example, a magnetic disk (including a floppy disk), an optical disk, a magnetic optical disk, or the like. The optical disk is composed of, for example, a CD-ROM (Compact Disk-Read Only Memory), a DVD (Digital Versatile Disk), Blu-ray (Registered Trademark) or the like. The magnetic optical disk is composed of an MD (Mini-Disk) or the like. The storage medium supplied to the user in a state incorporated in the device main body in advance is constituted by, for example, the ROM 112 of FIG. 2 in which the program is recorded.

It should be noted that, in the present specification, the steps defining the program recorded in the storage medium include not only the processing executed in a time series following this order, but also processing executed in parallel or individually, which is not necessarily executed in a time series.

The embodiments of the present invention described above are only illustrative, and are not to limit the technical scope of the present invention. The present invention can assume various other embodiments. Additionally, it is possible to make various modifications thereto such as omissions or replacements within a scope not departing from the spirit of the present invention. These embodiments or modifications thereof are within the scope and the spirit of the invention described in the present specification, and within the scope of the invention recited in the claims and equivalents thereof.

What is claimed is:

1. An exercise support device comprising:
a processor;
a memory; and
a sensor,
wherein the processor executes a program stored in the memory to perform operations comprising:
acquiring, from the sensor, at least one of biological information about a user related to an exercise and exercise information about the user related to the exercise; and
acquiring predetermined information in an outdoor activity involving a movement, on the basis of at least one of the biological information and the exercise information having been acquired,
wherein the processor is further configured to acquire at least one of the biological information and the exercise information resulting from an exercise performed by the user other than an exercise performed by the user as the movement in the outdoor activity.

2. The exercise support device according to claim 1, wherein the predetermined information is navigation information.

3. The exercise support device according to claim 1, wherein the processor is further configured to set a route with map information, and
wherein acquiring the predetermined information comprises acquiring the predetermined information about the route having been set.

4. The exercise support device according to claim 3, wherein setting the route comprises setting the route based on at least one of the biological information and the exercise information acquired having been acquired.

5. The exercise support device according to claim 3, wherein the processor is further configured to calculate a required time on the route having been set, on the basis of the biological information or the exercise information acquired having been acquired,
wherein acquiring the predetermined information comprises acquiring the required time having been calculated.

6. The exercise support device according to claim 1, wherein the processor is further configured to select one outdoor activity from a plurality of outdoor activities, and
wherein acquiring the predetermined information comprises acquiring the predetermined information on the basis of the outdoor activity having been selected.

7. The exercise support device according to claim 1, wherein acquiring at least one of the biological information and the exercise information comprises acquiring the biological information in at least one of daily exercise information and work out information of the user.

8. The exercise support device according to claim 7, wherein acquiring at least one of the biological information and the exercise information comprises identifying an exercise level of the user from at least one of the exercise information and the work out information, and
wherein acquiring the predetermined information comprises acquiring the predetermined information on the basis of the exercise level.

9. The exercise support device according to claim 7, wherein acquiring at least one of the biological information and the exercise information comprises acquiring the biological information or the exercise information of the user with a sensor fitted to the user or acquiring the biological information or the exercise information of the user by analyzing an image of the user sensed with a sensor.

10. The exercise support device according to claim 1, wherein the processor is further configured to determine an empirical value of the user on the basis of a history of an outdoor activity performed by the user in past, and
wherein acquiring the predetermined information comprises acquiring the predetermined information on the basis of the empirical value having been determined.

11. The exercise support device according to claim 1, wherein acquiring the predetermined information comprises acquiring the predetermined information in the outdoor activity of the user involving the movement on the basis of at least one of the biological information and the exercise information acquired for a plurality of other users.

12. An exercise support method executed by an exercise support device including a processor and a sensor, the exercise support method causing the processor to execute a program stored in a memory to perform operations comprising:
acquiring, from the sensor, at least one of biological information about a user related to an exercise and exercise information about the user related to the exercise; and
acquiring predetermined information in an outdoor activity involving a movement, on the basis of at least one of the biological information and the exercise information having been acquired,
wherein the processor is further configured to acquire at least one of the biological information and the exercise information resulting from an exercise performed by the user other than an exercise performed by the user as the movement in the outdoor activity.

13. The exercise support method according to claim 12, wherein the predetermined information is navigation information.

14. The exercise support method according to claim 12, wherein the processor is further configured to set a route with map information, and
wherein acquiring the predetermined information comprises acquiring the predetermined information about the route having been set.

15. The exercise support method according to claim 14, wherein setting the route comprises setting the route based on at least one of the biological information and the exercise information acquired having been acquired.

16. The exercise support method according to claim 14, wherein the processor is further configured to calculate a required time on the route having been set, on the basis of the biological information or the exercise information acquired having been acquired,
wherein acquiring the predetermined information comprises acquiring the required time having been calculated.

17. The exercise support method according to claim 12, wherein the processor is further configured to select one outdoor activity from a plurality of outdoor activities, and
wherein acquiring the predetermined information comprises acquiring the predetermined information on the basis of the outdoor activity having been selected.

18. The exercise support method according to claim 12, wherein acquiring at least one of the biological information and the exercise information comprises acquiring the biological information in at least one of daily exercise information and work out information of the user.

19. The exercise support method according to claim 18, wherein acquiring at least one of the biological information and the exercise information comprises identifying an exercise level of the user from at least one of the exercise information and the work out information, and
wherein acquiring the predetermined information comprises acquiring the predetermined information on the basis of the exercise level.

20. A non-transitory computer-readable storage medium storing a program that is executable by a computer that comprises a processor, the program being executable to cause the computer to perform operations comprising:
acquiring, from the sensor, at least one of biological information about a user related to an exercise and exercise information about the user related to the exercise; and
acquiring predetermined information in an outdoor activity involving a movement, on the basis of at least one of the biological information and the exercise information having been acquired,
wherein the processor is further configured to acquire at least one of the biological information and the exercise information resulting from an exercise performed by the user other than an exercise performed by the user as the movement in the outdoor activity.
